(19) United States
(12) Patent Application Publication
(10) Pub. No.: US 2006/0288450 A1
(43) Pub. Date: Dec. 21, 2006

(54) Plant N-acylethanolamine binding proteins

(75) Inventors: Kent D. Chapman, Denton, TX (US); Swati Tripathy, Denton, TX (US); Richard A. Dixon, Ardmore, OK (US)

Correspondence Address: FULBRIGHT & JAWORSKI L.L.P., 600 CONGRESS AVE., SUITE 2400, AUSTIN, TX, 78701, US

(73) Assignee: Samuel Roberts Noble Foundation

(21) Appl. No.: 11/176,594

(22) Filed: Jul. 7, 2005

Related U.S. Application Data
(60) Provisional application No. 60/585,892, filed on Jul. 7, 2004.

(51) Int. Cl. A01H 5/00; C07K 14/415; C07H 21/04; C12P 21/06; C12N 9/10; C12N 15/82; C12N 5/10; C12N 5/04

(52) U.S. Cl. 800/281; 435/69.1; 435/193; 435/468; 435/419; 530/370; 536/23.6

(57) ABSTRACT

The invention provides plant binding proteins of N-acylethanolamines. Also provided are constructs comprising coding sequences for the binding proteins, plants transformed therewith and methods of use thereof. The invention allows the modification of plant signaling by N-acylethanolamines. Such modification may be used to produce plants that are improved with respect to growth, seed germination, pathogen response and stress tolerance.

This application claims the priority of U.S. Provisional Patent Appl. Ser. No. 60/585,892, filed Jul. 7, 2004, the entire disclosure of which is specifically incorporated herein by reference. The government may own rights in this invention pursuant to grant number 99-35304-8002 from the USDA NRI.

### BACKGROUND OF THE INVENTION

1. Field of the Invention

The present invention relates generally to the field of molecular biology. More specifically, the invention relates to plant N-acylethanolamine binding proteins and methods of use thereof.

2. Description of the Related Art

N-acylethanolamines (NAEs) are endogenous constituents of plant and animal tissues. Like in animal cells, plant NAEs are derived from N-acylphosphatidylethanolamines (NAPEs), a minor membrane lipid constituent of cellular membranes, by the action of a phospholipase D (PLD) (Schmid et al., 1990; Chapman, 2000). Individual NAEs have been identified in plants as predominantly 16C and 18C species with N-palmitoylethanolamine (NAE 16:0) and N-linoleoylethanolamine (NAE 18:2) generally being the most abundant.

NAEs are released as endogenous ligands for cannabinoid (CB) receptors in vertebrates (Di marzo et al., 1994; Chapman et al., 1998; Hansen et al., 2000; Schmid et al., 2002). In animal systems, anandamide (NAE 20:4) is the endogenous signaling ligand for CB receptors and also activates vanilloid (VR1) receptors (Pertwee, 2001). Anandamide has varied physiological roles and functions in modulation of neurotransmission in the central nervous system (Wilson and Nicoll, 2002). Anandamide also functions as an endogenous analgesic (Pertwee, 2001) and appears to be involved in neuroprotection (Hansen et al., 2000; Van der Stelt et al., 2001). While a principal role for NAE20:4 as an endogenous ligand for cannabinoid receptors and endocannabinoid signaling is indicated, other types of NAEs as well as other fatty acid derivatives likely interact with this pathway and perhaps others directly or indirectly to modulate a variety of physiological functions in vertebrates (Lambert and Di Marzo, 1999; Lambert et al., 2002; Schmid and Berdyshev, 2002; Schmid et al., 2002).

The cannabinoid receptors and their endogenous ligands constitute the endocannabinoid signaling system in animal tissues and these ligands are antagonized specifically and potently by several commercially developed CB receptor analogs such as SR 144528 and AM 281, etc (Reggio, 1999; Khanolkar et al., 2000). Together the NAEs, their CB receptors and their competitive CB receptor agonists and antagonists are implicated as potential candidates for a variety of therapeutic applications (De Petrocellis et al., 2000; Straus, 2000).

Research during the last decade has suggested that NAE metabolism occurs in plants by pathways analogous to those in vertebrates and invertebrates (Chapman, 2000; Shrestha et al., 2002), pointing to the possibility that these lipids may be an evolutionarily conserved mechanism for the regulation of physiology in multicellular organisms. In plants, NAEs are present in substantial amounts in desiccated seeds (˜1 μg g⁻¹ fresh wt) and their levels decline after a few hours of imbibition (Chapman et al., 1999; Chapman, 2000). The occurrence of NAEs in seeds and their rapid depletion during seed imbibition suggests that these lipids may have a role in the regulation of seed germination (Chapman, 2000). Arabidopsis seedlings developed abnormal root growth when NAEs were elevated exogenously, pointing to a possible regulatory function for NAEs in seedling growth as well as seed germination (Blancaflor et al., 2003).

NAEs were released during plant pathogen-derived elicitor treatment and they activated defense gene (Phenylalanine-ammonia lyase) expression at submicromolar concentrations (Chapman et al., 1998; Shrestha et al., 2003; Tripathy et al., 1999). NAE-regulated defense gene expression in leaves was blocked by coadministration of mammalian CB receptor antagonist SR 144528, indicating the possible existence of animal-like endocannabinoid signaling system in plants (Tripathy et al., 1999).

NAEs have also been implicated in immunomodulation (Buckley et al., 2000), synchronization of embryo development (Paria and Dey, 2000), and induction of apoptosis (Sarker et al., 2000). These endogenous bioactive molecules lose their signaling activity upon hydrolysis by fatty acid amide hydrolase (FAAH).

The defense signaling activities by NAE 14:0 have been linked to a membrane-associated specific binding activity of [3H]NAE 14:0 in tobacco leaves. Similarly, high affinity, membrane-associated NAE-binding activities were identified in Arabidopsis and Medicago. The saturable, high affinity (in lower nanomolar range) binding activities of [3H]NAE 14:0 were potently challenged by CB receptor antagonist SR 144528 and AM 281 and provided further evidence for a CBlike receptor-mediated functioning of NAEs in plants (Tripathy et al., 2003a). The [3H]NAE 14:0 binding activity was successfully reconstituted in detergent-solubilized microsomal fraction of tobacco and Arabidopsis (Tripathy et al., 2003b). In order to understand the sequential events involve in NAE-mediated-signaling, it is essential to identify the molecular components.

While the foregoing studies have provided a further understanding of the metabolism of plant NAEs, the prior art has failed to provide genes encoding plant NAE binding proteins. Methods for modifying recognition of NAE signals in plants have thus been lacking. The identification of genes for such modification would allow the creation of novel plants with improved phenotypes and methods for use thereof. There is, therefore, a great need in the art for the identification of nucleic acid sequences encoding plant NAE binding proteins.

### SUMMARY OF THE INVENTION

In one aspect, the invention provides an isolated nucleic acid sequence encoding a plant NAE binding protein. In certain aspects of the invention, the plant NAE binding protein may be from a species selected from the group consisting of: Arabidopsis thaliana, barley, sunflower, loblolly pine, maize, potato, rice, rye, sugarcane, sorghum, soybean, tomato, wheat and Medicago truncatula. In one embodiment, the nucleic acid is further defined as selected from the group consisting of: (a) a nucleic acid sequence encoding the polypeptide of SEQ ID NO:2 or SEQ ID NO:4; (b) a nucleic acid sequence comprising the sequence of SEQ ID NO:1 or SEQ ID NO:3; and (c) a nucleic acid sequence hybridizing to SEQ ID NO 1 or SEQ ID NO:3 under conditions of 5×SSC, 50% formamide and 42° C. In another aspect, the invention provides an isolated nucleic acid sequence encoding a plant NAE binding protein selected from the group consisting of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 and SEQ ID NO:29. Also provided by the invention are nucleic acids encoding the polypeptides encoded by these sequences.

In another aspect, the invention provides a recombinant vector comprising an isolated polynucleotide of the invention. In certain embodiments, the recombinant vector may further comprise at least one additional sequence chosen from the group consisting of: a regulatory sequence, a selectable marker, a leader sequence and a terminator. In further embodiments, the additional sequence is a heterologous sequence and the promoter may be developmentally-regulated, organelle-specific, inducible, tissue-specific, constitutive, cell-specific, seed specific, or germination-specific promoter. The recombinant vector may or may not be an isolated expression cassette.

In still yet another aspect, the invention provides an isolated polypeptide comprising an NAE binding protein sequence provided by the invention, or a fragment thereof having NAE binding protein activity.

In still yet another aspect, the invention provides a transgenic plant transformed with a selected DNA comprising a nucleic acid sequence of the invention encoding an NAE binding protein. The transgenic plant may be a monocotyledonous or dicotyledonous plant. The plant may also be an RO transgenic plant and/or a progeny plant of any generation of an RO transgenic plant, wherein the transgenic plant has inherited the selected DNA from the RO transgenic plant. The invention further provides a seed of a transgenic plant of the invention, wherein the seed comprises the selected DNA, as well as a host cell transformed with such a selected DNA. The host cell may express a protein encoded by the selected DNA. The cell may have inherited the selected DNA from a progenitor of the cell and may have been transformed with the selected DNA. The cell may be a plant cell.

In still yet another aspect, the invention provides a method of altering the N-acylethanolamine perception of a plant comprising up- or down-regulating NAE binding protein in the plant. In one embodiment, the method comprises down-regulating NAE binding protein in the plant, wherein the N-acylethanolamine signaling is decreased as a result of the down-regulating. In another embodiment of the invention, the method comprises up-regulating NAE binding protein in the plant, wherein the physiological effects of N-acylethanolamine signaling in the plant is increased as a result of the up-regulating.

In still yet another aspect, the invention provides a method of modulating the growth of a plant or part thereof, comprising up- or down-regulating NAE binding protein in the plant or part thereof. In one embodiment, the method comprises up-regulating NAE binding protein in the plant, wherein the growth of the plant is decreased as a result of the up-regulating. In another embodiment of the invention, the method comprises down-regulating NAE binding protein in the plant, wherein the growth of the plant is increased as a result of the down-regulating.

In still yet another aspect, the invention provides a method of modulating stress tolerance in a plant or part thereof, comprising up- or down-regulating NAE binding protein in the plant or part thereof. In one embodiment, the method comprises down-regulating NAE binding protein in the plant, wherein the stress tolerance of the plant is decreased as a result of the down-regulating. In another embodiment of the invention, the method comprises up-regulating NAE binding protein in the plant, wherein the stress tolerance of the plant is increased as a result of the up-regulating.

In still yet another aspect, the invention provides a method of modulating pathogen perception in a plant or part thereof, comprising up- or down-regulating NAE binding protein in the plant or part thereof. In one embodiment, the method comprises down-regulating NAE binding protein in the plant, wherein the pathogen perception of the plant is decreased as a result of the down-regulating. In another embodiment of the invention, the method comprises up-regulating NAE binding protein in the plant, wherein the pathogen perception of the plant is increased as a result of the up-regulating.

In a method of the invention, up-regulating may comprise introducing a recombinant vector of the invention into a plant. Down-regulating may comprise introducing a recombinant vector into a plant, wherein the nucleic acid or antisense or RNAi oligonucleotide thereof is in antisense orientation relative to the heterologous promoter operably linked thereto. Down-regulating may also comprise mutating an endogenous NAE binding protein coding sequence. The vector may be introduced by plant breeding and/or direct genetic transformation.

In still yet another aspect, the invention provides a method of making food for human or animal consumption comprising: (a) obtaining the plant of the invention; (b) growing the plant under plant growth conditions to produce plant tissue from the plant; and (c) preparing food for human or animal consumption from the plant tissue. In the method, preparing food may comprise harvesting plant tissue. In certain embodiments, the food is starch, protein, meal, flour or grain.

The use of the word “a” or “an” when used in conjunction with the term “comprising” in the claims and/or the specification may mean “one,” but it is also consistent with and encompasses the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1. Analysis of specific binding of [³H]NAE 14:0 to DDM solubilized (0.2 mM) microsomes (25 μg of protein) isolated from A. thaliana (ecotype Columbia) leaves (150,000×g) with a concentration ranging from 10 to 80 nM. Specific binding was determined by subtracting nonspecific binding (binding in the presence of 1,500× nonradioactive NAE 14:0) values from total radioligand binding values in duplicate assays. Binding affinity and Bₘₐₓ of [³H]NAE 14:0 was estimated by fitting the saturation binding data to nonlinear regression analysis for a one-site binding equation using Prism 3.0, Graphpad software, San Diego.

FIG. 2. Flowchart showing the bioinformatic tools used for identifying candidate NAE binding protein(s) in A. thaliana using mammalian cannabinoid and vanilloid NAE receptor proteins

FIG. 3. At1g26440 amino acid sequence deduced from cDNA (YAP105T7), topology prediction (hmmtop *), and motif organization (*Tusnády and Simon, 1998; Tusnády and Simon, 2001). Bold—Transmembrane segment; Italics—A[DLN].L.S[ITV].FHWY (putative NAE binding): 10⁻²², IBM Pattern search; Underline—MEME/MAST common motif: 10⁻³⁴; Bold italics—IVT motif [ITV]. [ITV].L.I.[FHWY]: 10⁻¹⁷, IBM Pattern search.

FIG. 4 Full length cDNAs synthesized through RT-PCR on Arabidopsis total RNA using gene specific primers to At1g26440 were cloned into pTrc His₂ expression vector and the recombinant protein over expressed in E. coli Top10 competent cells (Invitrogen, Cat. No. K4400-01 and K4400-40). Positive clones selected on amp-glucose agar plates were analyzed for producing NAE 14:0 binding protein by using [³H]NAE 14:0 binding assay (Tripathy et al., 2003). Bacterial lysate (L) prepared from pelleted positive clones induced with IPTG for recombinant protein production was used as the protein source. T10-1, T-10-2, T10-3: Positive clones carrying A. thaliana NAE-binding protein (At1g26440); T10-4: Negative clone carrying the gene sequence in reverse orientation; T10-0: Control Top10 cells. Binding assay was performed on cell lysate (L) and the affinity purified (ProBond purification system, Invitrogen) recombinant protein (P) fraction. Histograms represent binding activity (from two studies with four replicates each).

FIG. 5 Saturation binding of [³H]NAE 14:0 to a candidate NAE binding protein (At1g26440) expressed in E. coli Top10 cell lysate. The candidate NAE binding protein was a full-length, in-frame cDNA. [³H]NAE 14:0 binding assay was carried out with DDM (0.2 mM) solubilized cell lysate. The Bₘₐₓ and K_{d} values were estimated by fitting specific binding values to a nonlinear regression analysis for a one-site binding equation (Prism, 3.0, GraphPad software, Sand Diego).

FIG. 6 Saturation binding of [³H]NAE 14:0 to a candidate NAE binding protein (At1g26440) expressed and purified from E. coli Top10 cell lysate using ProBond purification system (Invitrogen, San Diego). The candidate NAE binding protein was a full-length, in-frame cDNA. Proteins were affinity purified from DDM (0.2 mM) solubilized cell lysate fractionated on Probond with protein produced from the full length in frame cDNA expressed in E. coli Top10 cells. [3 H]NAE 14:0 binding assay was carried out in DDM (0.2 mM).

FIG. 7 Microsomes from A. thaliana leaves and affinity purified (P) protein from T10-1 cell lysate were incubated with [³H]NAE 14:0 (50 nM) in the presence and absence of CB receptor antagonist SR144528 (5 nM) to compare the influence on [3H]NAE binding. Values represent means of triplicate assays from an individual experiment reproduced twice.

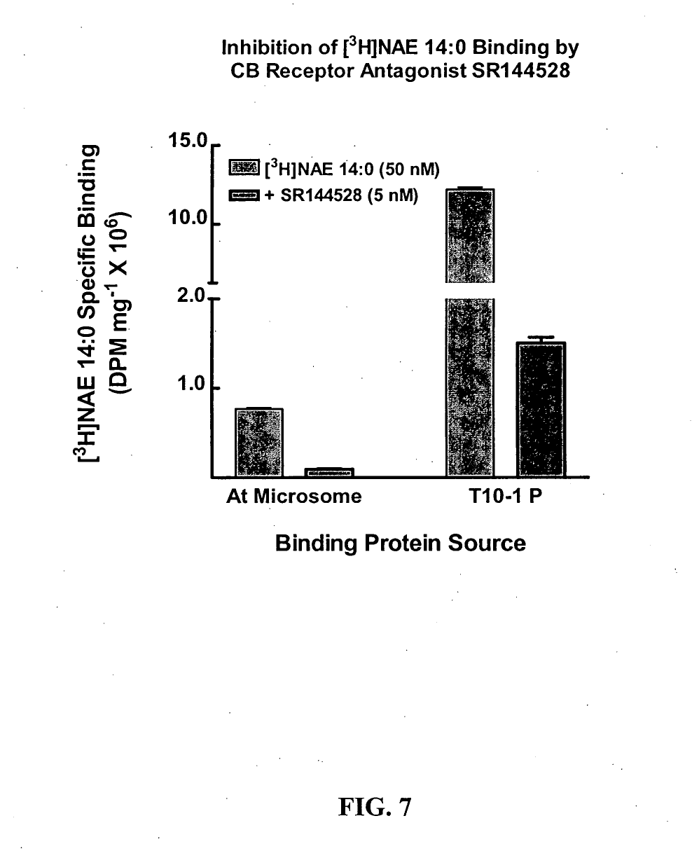

FIG. 8 Microsomes were isolated from leaves of A. thaliana ecotype, columbia (wild type control) and two mutants with a T-DNA inserted into At1g26440 (SALK-044810). Both microsomes and DDM-solubilized (0.2 mM) microsomes (Tripathy et al., 2003) were incubated with [3H]NAE 14:0 (25 nM) and specific binding activity was estimated by subtracting [3H]NAE 14:0 binding in the presence of excess of NAE 14:0 (1500×) from [3H]NAE 14:0 binding alone. Values represent [3H]NAE 14:0 binding activity from means of (triplicate assays) an individual experiment reproduced two times. T3 seed of mutants were obtained from the ABRC stock center and homozygous individuals were identified by PCR according to the SALK web site. Plants were grown under 10-h photoperiod and 23° C. Plants were harvested at approximately 6 weeks old.

### DETAILED DESCRIPTION OF THE INVENTION

The invention overcomes the limitations of the prior art by providing, for the first time, isolated nucleic acid encoding plant N-acylethanolamines (NAE) binding proteins. The invention is significant in that the biological activity of NAEs are mediated by NAE binding proteins and nucleic acids encoding plant NAE binding proteins had not been previously isolated. NAEs are a group of bioactive fatty acid derivatives that have a variety of important physiological activities. The invention therefore allows, for the first time, the creation of transgenic plants with modified NAE binding protein expression comprising improved phenotypes.

In accordance with the invention introduction of a heterologous NAE binding protein coding sequence may be used to increase NAE binding protein expression and up-regulate NAE signaling in the plant. Similarly, the invention now allows decreasing NAE signaling by down-regulating NAE binding protein in a plant or any parts thereof, including a given cell, for example, by mutating a native NAE binding protein coding sequence, or using antisense, RNAi or any other desired technique known in the art using the nucleic acid sequences provided herein.

Among the important physiological roles identified for NAEs in plants is perception of fungal elicitors. In particular, levels of endogenous NAE 14:0 are elevated 10-50 fold in leaves of tobacco plants following fungal elicitation (Tripathy et al., 1999). These NAE levels measured endogenously were shown sufficient to activate downstream defense gene expression in plants (Tripathy et al., 1999), and mammalian cannabinoid receptor antagonists abrogated the downstream response (Tripathy et al., 2003). A high-affinity NAE14:0-binding protein was identified in plant membranes and was indicated to mediate the NAE activation of defense gene expression (Tripathy et al., 2003). Therefore, one application of the current invention is in the alteration of plant perception to pathogens. By up-regulating NAE binding protein, increased perception of pathogen elicitors may be obtained. Similarly, host cell defense mechanisms may be decreased with the invention by the down-regulation of NAE binding protein. The foregoing may be achieved, for example, using inducible promoters activated by one or more pathogen elicitor, or using constitutive or other desired regulatory elements.

NAEs (primarily C12, C16 and C18 types) have also been shown to be present in high levels in desiccated seeds of higher plants, but metabolized rapidly during the first few hours of seed imbibition/germination (Chapman et al., 1999), in part by an amidohyrolase-mediated pathway (Shrestha et al., 2002). The transient changes in NAE content indicate a role in seed germination. In fact, Arabidopsis seedlings germinated and grown in the presence of exogenous NAE exhibit dramatically altered developmental organization of root tissues. An important role in seed germination and cell division in general has therefore been indicated. This is supported by evidence in mammalian cells that NAEs can stimulate apoptosis. Therefore, it may also be desired in accordance with the invention to modulate NAE levels to modulate cell division. By decreasing NAE binding protein, a corresponding increase in cell division may be obtained. This may be desirable, for example, for the creation of plants having shortened stature, or, through use of temporally- and/or developmentally-regulated heterologous promoter, for modulating growth at a given time or stage of development. Seed germination may also be modified. Alternatively, growth of plants may be decreased by increasing NAE binding protein expression.

In certain embodiments of the invention, a NAE binding protein may be modulated in conjunction with one or more coding sequences that alter NAE content. For example, in plants fatty acid amide hydrolase (FAAH) catalyzes the hydrolysis of N-acylethanolamines (NAEs), which are endogenous constituents of plant and animal tissues. The hydrolysis terminates biological activities of NAEs. Therefore, FAAH may be modified in conjunction with a NAE binding protein. By increasing FAAH expression to decrease NAE levels and down-regulating NAE binding protein, further decreases in NAE signaling may be obtained. Alternatively, NAE binding proteins may be expressed heterologously in conjunction with the down-regulation of FAAH to increase NAE signaling. One example of an FAAH coding sequence that could be used with the invention is a nucleic acid encoding the polypeptide of SEQ ID NO:43, for example, the nucleic acid sequence of SEQ ID NO:42. Techniques for the transgenic modification of FAAH are described in U.S. patent application Ser. No. 10/862,063, application pub. No.20050028233.

### I. Plant Transformation Constructs

In one embodiment of the invention plant transformation constructs are provided encoding one or more NAE binding protein coding sequence. An exemplary coding sequence for use with the invention is an Arabidopsis thaliana NAE binding protein comprising the polypeptide sequence of SEQ ID NO:2. Such a coding sequence may comprise the Arabidopsis NAE binding protein nucleic acid sequence of SEQ ID NO:1.

Also provided by the invention are orthologous NAE binding proteins sequences from plant species other than Arabidopsis. In certain embodiments of the invention the orthologous sequences are from barley, sunflower, loblolly pine, maize, potato, rice, rye, sugarcane, sorghum, soybean, tomato, wheat or Medicago truncatula. Examples of such sequences are given in SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 and SEQ ID NO:29. One embodiment of the invention therefore provides a recombinant vector comprising one or more of the foregoing sequences, including all possible combination thereof, as well as plants transformed with these sequences. Also provided by the invention are nucleic acids encoding the polypeptides encoded by these sequences.

Sequences that hybridize to any of the sequences provided by the invention under stringent conditions are also provided. An example of such conditions is 5×SSC, 50% formamide and 42° C. It will be understood by those of skill in the art that stringency conditions may be increased by increasing temperature, such as to about 60° C or decreasing salt, such as to about 1×SSC, or may be decreased by increasing salt, for example to about 10×SSC, or decreasing temperature, such as to about 25 ° C.

Nucleic acids provided by the invention include those encoding active NAE binding protein fragments. Those of skill in the art will immediately understand in view of the disclosure that such fragments may readily be prepared by placing fragments of NAE binding protein coding sequences in frame in an appropriate expression vector, for example, comprising a plant promoter. Using the assays described in the working examples, NAE binding protein activity can be efficiently confirmed for any given fragment. Fragments of nucleic acids may be prepared according to any of the well known techniques including partial or complete restriction digests and manual shearing.

Sequences provided by the invention may be defined as encoding a functional (e.g., active) NAE binding protein. In certain further aspects of the invention, a plant NAE binding protein may be characterized as from a monocotyledonous or dicotyledonous plant. Coding sequences may be provided operably linked to a heterologous promoter, in sense or antisense orientation. Expression constructs are also provided comprising these sequences, including antisense and RNAi oligonucleotides thereof, as are plants and plant cells transformed with the sequences.

The construction of vectors which may be employed in conjunction with plant transformation techniques using these or other sequences according to the invention will be known to those of skill of the art in light of the present disclosure (see, for example, Sambrook et al., 1989; Gelvin et al., 1990). The techniques of the current invention are thus not limited to any particular nucleic acid sequences.

One important use of the sequences provided by the invention will be in the alteration of plant phenotypes by genetic transformation with NAE binding protein coding sequences. The NAE binding protein coding sequence may be provided with other sequences for efficient expression as is known in the art. One or more selectable marker genes may be co-introduced into a plant with a nucleic acid provided by the invention.

The choice of any additional elements used in conjunction with a NAE binding protein coding sequence will often depend on the purpose of the transformation. One of the major purposes of transformation of crop plants is to add commercially desirable, agronomically important traits to the plant, as described above.

Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. In one embodiment of the invention, the native promoter of a NAE binding protein coding sequence is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is contemplated that vectors for use in accordance with the present invention may be constructed to include an ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). The use of an enhancer element, such as the ocs element and particularly multiple copies of the element, may act to increase the level of transcription from adjacent promoters when applied in the context of plant transformation.

It is envisioned that NAE binding protein coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue.

B. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of a NAE binding protein coding sequence is used. Alternatively, a heterologous 3′ end may enhance the expression of sense or antisense NAE binding protein coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms selectable or screenable markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228). The gene that encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### II. Antisense and RNAi Constructs

Antisense and RNAi treatments represent one way of altering NAE binding protein activity in accordance with the invention. In particular, constructs comprising a NAE binding protein coding sequence, including fragments thereof, in antisense orientation, or combinations of sense and antisense orientation, may be used to decrease or effectively eliminate the expression of NAE binding protein in a plant. Accordingly, this may be used to “knock-out” the function of an NAE binding protein coding sequence or homologous sequences thereof.

Techniques for RNAi are well known in the art and are described in, for example, Lehner et al., (2004) and Downward (2004). The technique is based on the fact that double stranded RNA is capable of directing the degradation of messenger RNA with sequence complementary to one or the other strand (Fire et al., 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the invention, such an oligonucleotide may comprise any unique portion of a nucleic acid sequence provided herein. In certain embodiments of the invention, such a sequence comprises at least 18, 30, 50, 75 or 100 or more contiguous nucleic acids of the nucleic acid sequence of SEQ ID NO:1, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs will include regions complementary to intron/exon splice junctions. Thus, it is proposed that a preferred embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., ribozyme; see above) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

### III. Methods for Genetic Transformation

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

A. Agrobacterium-mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et aL., 1991), maize (Bhattacharjee et al., 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

E. Tissue Cultures

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. Bactoagar, Hazelton agar, Gelrite, and Gelgro are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al. (1975) and MS media (Murashige and Skoog, 1962).

### IV. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Libertym also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al, 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m-2 s-i of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCRTM; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### V. Breeding Plants of the Invention

In addition to direct transformation of a particular plant genotype with a construct prepared according to the current invention, transgenic plants may be made by crossing a plant having a selected DNA of the invention to a second plant lacking the construct. For example, a selected NAE binding protein coding sequence can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants.

As used herein the term “progeny” denotes the offspring of any generation of a parent plant prepared in accordance with the instant invention, wherein the progeny comprises a selected DNA construct. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. Definitions

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Identification of A NAE Binding Protein Coding Sequence

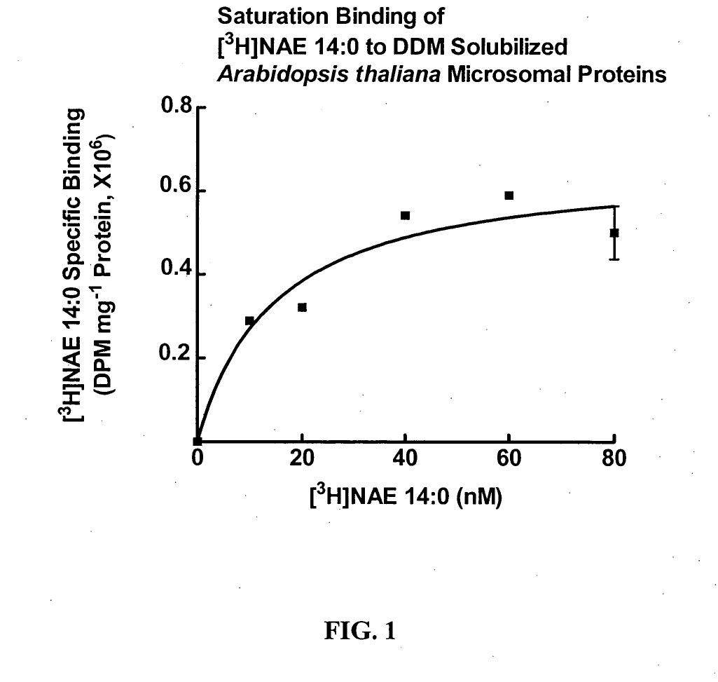

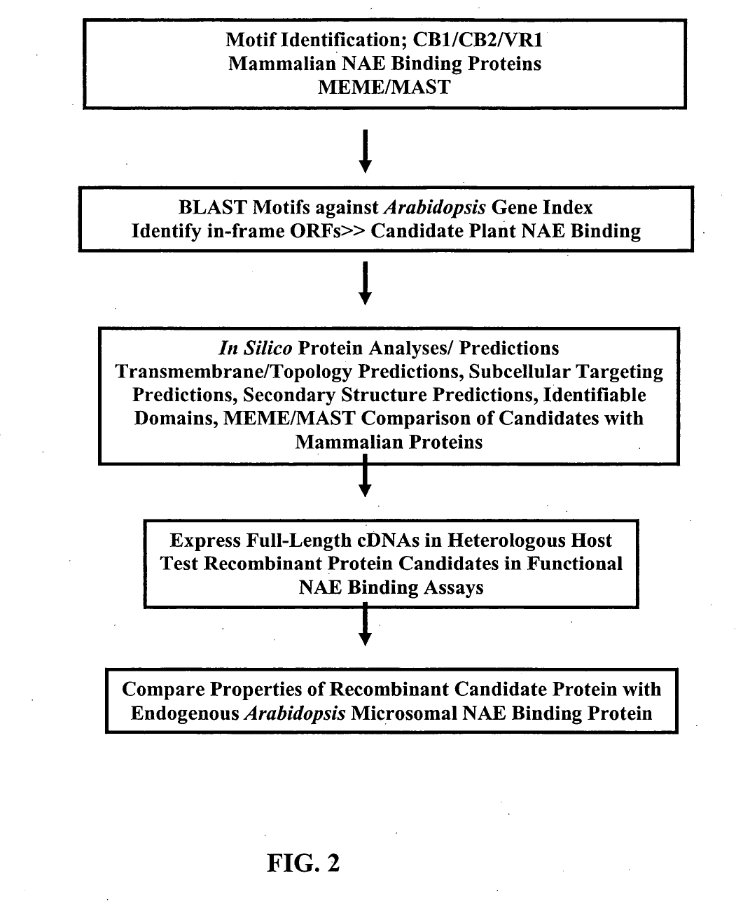

N-acylethanolamines (NAEs) bind with high affinity to membranes isolated from various plant tissues (Tripathy et al., 2003). This NAE binding activity was solubilized from Arabidopsis leaf microsomes in dodecylmaltoside (DDM) while retaining the functional activity (FIG. 1). The functional binding assay was used to employ strategies to identify DNA sequences encoding functional NAE binding proteins from plants (FIG. 2).

A bioinformatics strategy was initially followed to identify candidate Arabidopsis NAE binding proteins, with sequences from NAE binding proteins in vertebrates as a reference point. Fifteen motifs were identified by analysis of three different proteins known to bind with high affinity to NAEs; the rat cannabinoid receptor type 1 (CB1), mouse cannabinoid receptor type 2 (CB2) and human vannilloid receptor type 1 (VR1) (Table 1). These motifs were identified using computational methods described at www.meme.sdsc.edu (Bailey and Elkan, 1994; Bailey and Gribskov, 1998), and were used to query the Arabidopsis gene index for proteins with matches. Ten Arabidopsis candidate nucleotide sequences encoding proteins with in-frame matches were identified with similarity to one or more motif from Table 1 and are listed in Table 2.

TABLE 1

Motifs identified by MEME/MAST analyses
(meme.sdsc.edu) comparing Rat CB1, Mouse
CB2, and Human VR1 receptors (mammalian
NAE binding proteins) (SEQ ID NOs:30-41).

| | | |
|---|---|---|
| MOTIF | WIDTH | BEST POSSIBLE MATCH |

| | | |
|---|---|---|
| 1 | 50 | ICWGPLLAIMVHDVFGKMNDQIKTAFAFCSMLCLINS |
| | | MVNPIIYALRSGD |
| 2 | 41 | SRRLRCKPSYHFIGSLAGADFLGSVIFVCSFVDFHVF |
| | | HGKD |
| 3 | 50 | CSDIFPLIPEDYLMFWIGFTAILFSGIIYTYGYILWK |
| | | AHQHVVRMIQHQD |
| 4 | 50 | KIGGVTMSFTASVGSLFLTAIDRYICIHYPPAYKRIV |
| | | TRPKAVVAFCIMW |
| 5 | 50 | EGIQCGENFNPMECYMILNPSQQLAIAVLCTTLGTFS |
| | | VLENLAVLCIILH |
| 6 | 50 | FRTITTDLLYVGSNDIQYEDMKGDMASKLGYFPQKFP |
| | | LSSFRGDPFQEKM |
| 7 | 21 | RQVPGPDQMRMDIRLAKTLGL |
| 8 | 42 | DCQHKHANNAGNVHRAAESCIKSTVKIAKVTMSVSTD |
| | | TSAEA |
| 9 | 40 | IRSAAQHCLIGWKKCVQGLGPEGKEEAPRSSVTETEA |
| | | DGK |
| 10 | 11 | PLMGWNCCPPP |
| 11 | 21 | HAFRSMFPSCEGTAQPLDNSM |
| 12 | 21 | PAEQVNITEFYNKSLSSFKEN |
| 13 | 15 | DRWCFQQEEVQWKHW |
| 14 | 8 | IHTTEDGK |
| 15 | 11 | MKSILDGLADT |

TABLE 2

Arabidopsis NAE binding protein candidates.
Identified as in-frame matches of predicted
translation products with CB motifs. Iden-
tified by WU-BLAST (no filter, expect 10000,
BLOSUM62) against Arabidopsis genome.

| Sequences producing | | |
|---|---|---|
| High-scoring Segment | | |
| Pairs | Frame | Score |
| arab\|BE529969 | +1 | 58 |
| arab\|AI998812 | −3 | 70 |
| arab\|TC122878 Contains | +3 | 61 |
| similarity to an A3 protein | | |
| arab\|TC105624 receptor-like | −1 | 69 |
| protein kinase | | |
| arab\|TC127108 | +2 | 68 |
| putative protein | | |
| Arabidopsis thaliana] | | |
| arab\|TC123338 | −2 | 65 |
| Highly similar to | | |
| auxin-induced protein. | | |
| arab\|TC122243 | +2 | 65 |
| putative protein | | |
| Arabidopsis thaliana] | | |
| arab\|TC104138 receptor | +3 | 65 |
| protein kinase-like protein | | |
| arab\|TC110443 FRO1-like | −1 | 63 |
| protein / NADPH oxidase | | |
| arab\|TC104832 CDS | −1 | 62 |

Mammalian NAE (NAE 20:4) binding protein sequences (CB1, CB2, VR1) from databases were subjected to multiple sequence alignments for extraction of conserved motifs from blocks of sequences using the combined web resource MEME/MAST analyses tools. These identified a range of most possible ungapped motif widths. Further analysis also identified the number times these motifs were present in each receptor sequences. The motifs identified are given in Table 3.

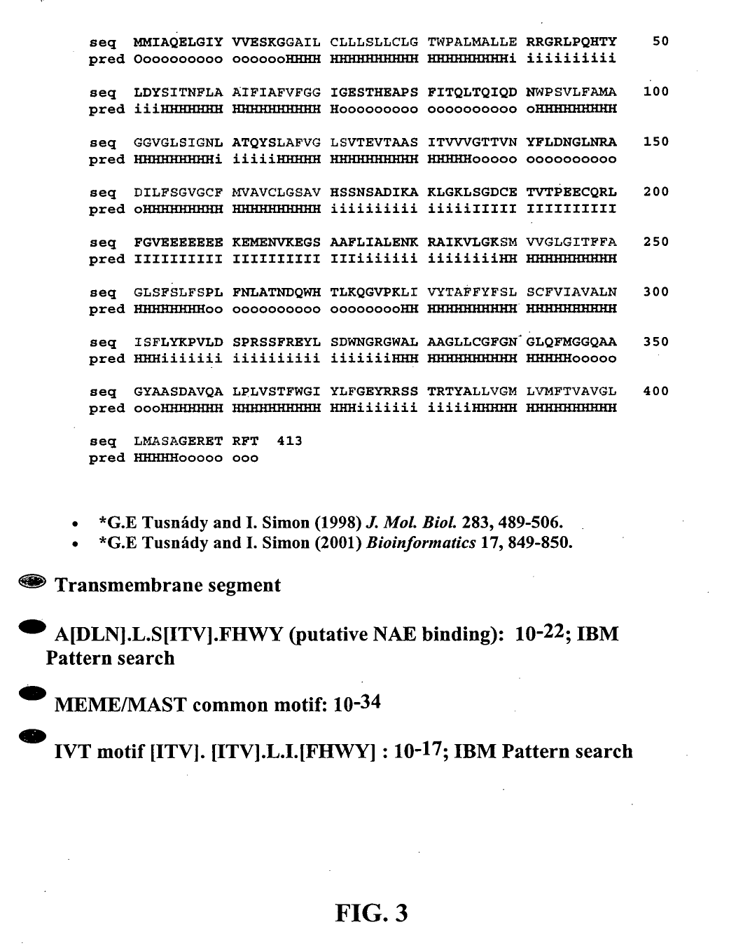

An EST (YAP105T7) corresponding originally to the TC122878 annotated at www.tigr.org, was obtained from the Arabidopsis Biological Resource Center, and sequenced completely on both strands. The nucleotide sequence was 1,459 bp in length and was predicted to encode a protein of 413 amino acids (SEQ ID NO:2; FIG. 3), and was derived from the gene, designated At1g26440. The protein primary sequence was subjected to various computer-based analyses, including topology predictions (hmmtop), secondary structure predictions (hmmtop), subcellular targeting (pSORT) and motif elicitation (MEME/MAST). The protein was predicted to have 10 transmembrane segments and three domains similar to CB1/CB2 receptors (FIG. 3). The protein was also predicted to be localized in the secretory pathway-ER, plasma membrane, or Golgi membranes.

TABLE 3

Motifs identified by MEME and MAST analysis
as common to vertebrate CB receptors and
Arabidopsis At1g26440 (SEQ ID NOs:44-52).

| | | |
|---|---|---|
| Protein | AA position | Sequence |

| | | |
|---|---|---|
| At1g26440 | (319) | YLSDWNGRGWALAAGLL |
| | | CGFGNGLQFMGGQAAGY |
| | | AASDAVQALPLVST |
| Mouse CB2 | (190) | YLLGWLLFIAILFSGII |
| | | YTYGYVLWKAHRHVATL |
| | | AEHQDRQVPGIARM |
| Zebrafish | (277) | YLMFWIGVTSILLLFIV |
| CB1 | | YAYMYILWKAHSHAVRM |
| | | LQRGTQKSIIIQST |

### Example 2

### Confirmation of NAE Binding Protein Identity

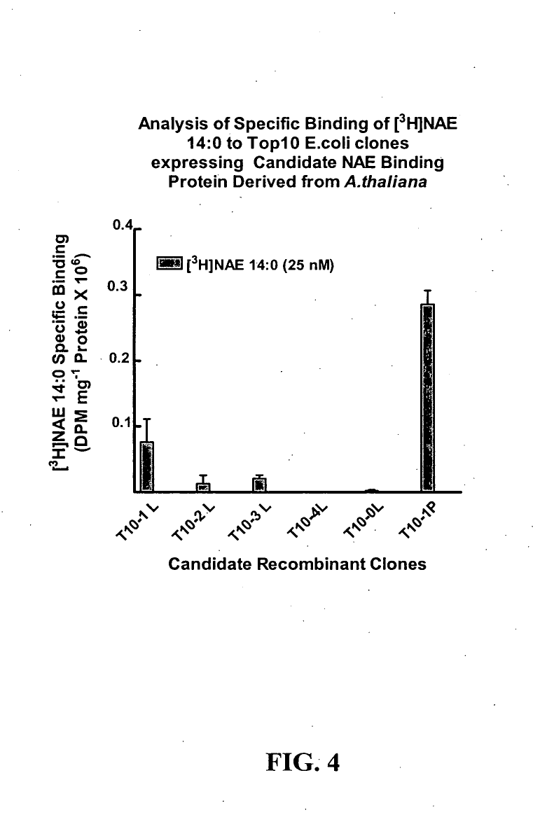

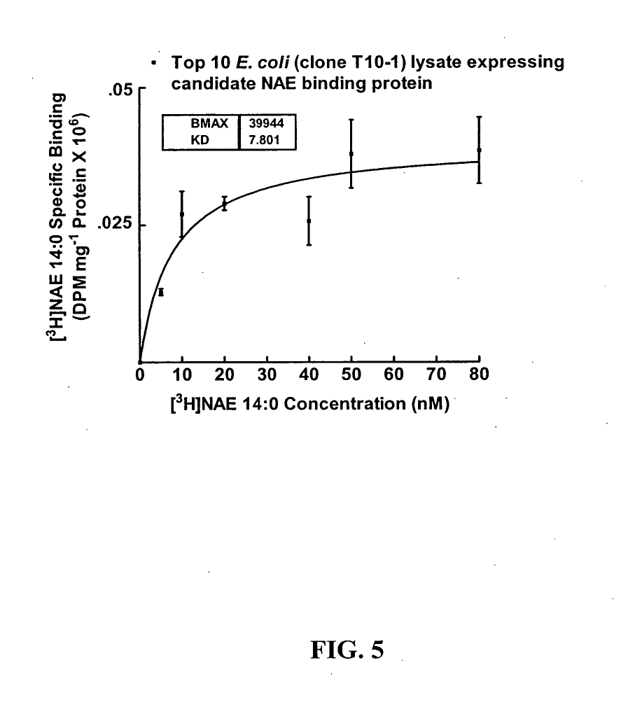

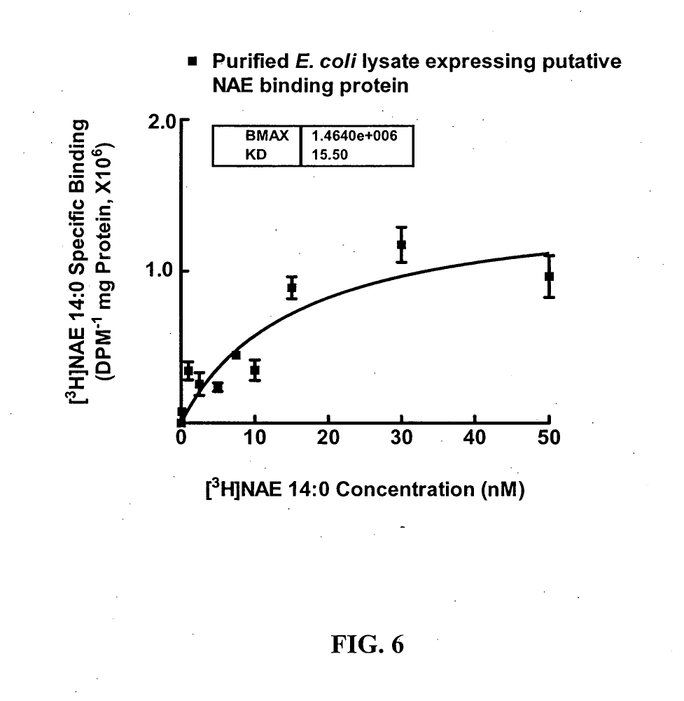

The cDNA encoding the putative Arabidopsis NAE binding protein was subcloned into a bacterial expression vector (pTrcHIS) as a His-tagged fusion. Lysates from bacterial cell lines expressing this cDNA in the correct orientation (T10-IL, T10-2L, T10-3L) exhibited specific NAE14:0 binding activity, whereas lysates from cells without this cDNA (T10-0L), or with the cDNA in the reverse orientation (T10-4L) showed no NAE binding activity (FIG. 4). This NAE binding activity was enriched when lysates in dodecylmaltoside detergent were subjected to Ni+ affinity chromatography (T10-1P). The recombinant protein expressed in E. coli exhibited saturation binding with respect to NAE14:0 and showed high affinity for NAE 14:0 similar to that estimated in detergent-solubilized Arabidopsis microsomes (compare FIGS. 5, 6 to FIG. 1). As an index of specificity, binding experiments in the presence of 5 nM SR144528, an antagonist of the vertebrate CB receptor, showed diminished NAE14:0 binding for both Arabidopsis microsomal protein and the recombinant At1g26440 gene product expressed in E. coli. Higher concentrations of SR144528 eliminated NAE specific binding.

The results were consistent with the notion that this Arabidopsis protein shares functional similarity with NAE binding proteins of vertebrates, and indicates that the sequence encodes a functional NAE binding protein. This was the first report of a nucleotide sequence from plants for an NAE binding protein. It was therefore indicated based on previous pharmacological experiments (Tripathy et al., 2003) that the gene/protein is involved NAE-regulated gene expression in plants.

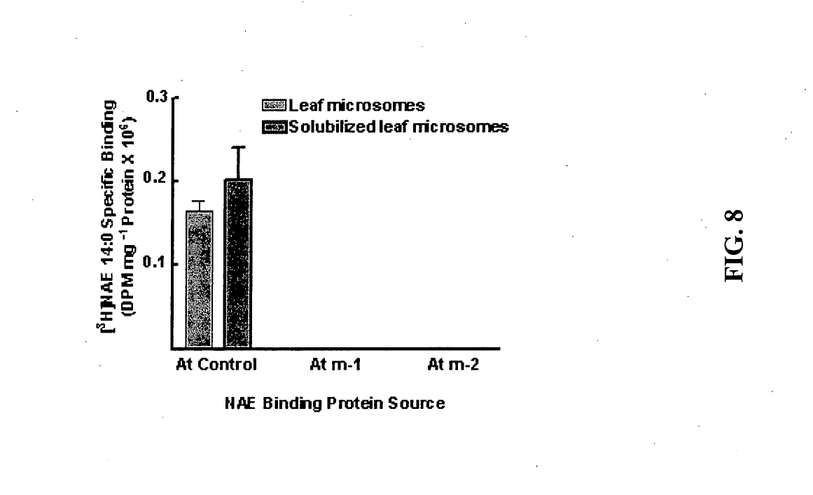

Further confirmation of the identity of the NAE binding protein was provided by the identification of two T-DNA insertional mutants in the corresponding locus. The mutant was identified with the Salk functional genomics program. Both microsomes and DDM solubilized (0.2 mM) microsomes (Tripathy et al., 2003) were incubated with [3H]NAE 14:0 (25 nM) and specific binding activity of the mutants was estimated by subtracting [3H]NAE 14:0 binding in the presence of excess of NAE 14:0 (1500×) from [3H]NAE 14:0 binding alone. The results demonstrated no NAE binding activity. The results are given in FIG. 8 and show [3H]NAE 14:0 binding activity (means of triplicate assays from an individual experiment reproduced two times).

### Example 3

### Radioligand Binding Assays

[³H]NAE 14:0 radioligand was synthesized from [9,10-³H(N)]myristic acid (PerkinElmer Life Sciences, Boston) in a modified two step reaction (Hillard et al., 1995; Devane et al., 1992). In step one, acylchloride is prepared from [3 H] 14:0 in the presence of dichloromethane, oxalyl chloride and dimethylformamide on ice and in step two the acylchloride formed was converted into N-acylethanolamine ([³H]NAE 14:0) in the presence of excess ethanolamine. The [³H]NAE 14:0 formed was further purified and quantified by separating on thin layer chromatography (TLC) and radiometric scanning respectively (Tripathy et al., 2003).

Binding activity of [³H]NAE 14:0 was carried out with DDM-solubilized microsomes from Arabidopsis (ecotype, columbia) leaves (Tripathy et al., 2003b) or recombinant protein produced in E. coli and solubilized in DDM. Briefly, radioligands were incubated with solubilized protein (10-20 μg) alone (total binding) and with excess of the cold ligand (non specific binding) in separate wells of a multiscreen Whatman filtration system having BC Durapore 1.2-mM filters (Millipore, Bedford, Mass.). Unbound ligands were washed off with ice cold binding buffer (75 mM potassium-phosphate buffer, pH 7.2, 300 mM sucrose, 7.5 mM KCl, 0.75 mM EDTA, 0.75 mM EGTA, 0.5 mM ascorbate, 5 mM DTT, and 1.5% BSA) under vacuum and frozen filters were excised into scintillation cocktail for radioactivity estimation.

### Example 4

### Identification of Plant NAE Binding Protein Orthologs

Using the Arabidopsis NAE binding protein as a reference point, a search was carried out to identify orthologous NAE binding protein coding sequences. Orthologous sequences were identified using the Washington University BLAST (WU-BLAST) program. The analysis was carried out on the plant gene indices assembled at www.tigr.org (including partial length sequences), with the exception of Medicago truncatula, which was sequenced from an EST in a Noble Foundation collection.

Sequences identified by the analysis are provided in Table 4. The best fits as an ortholog are in bold. Corresponding M. truncatula nucleic acid and polypeptide sequences are given in SEQ ID NOs:3 and 4, respectively. The remaining nucleic acids listed in Table 4 are given in SEQ ID NOs:5-29, respectively.

TABLE 4
Plant NAE binding protein orthologs of the Arabidopsis
NAE binding protein identified by WU-Blast analysis

| Identifier plant | % aa identity | match length | p-value |
|---|---|---|---|
| Medicago EST (NF018F12EC)** | 65% | 403 aa | 1e−145 |
| rice\|TC216007 Oryza sativa | 60% | 414 aa | 2.1e−127 |
| rice\|TC232971 Oryza sativa | 54% | 417 aa | 5.3e−121 |
| maize\|TC226528 Zea mays | 63% | 192 aa | 1.4e−82 |
| maize\|CF624774 Zea mays | 71% | 149 aa | 5.6e−56 |
| barley\|TC120475 Hordeum vulgare | 59% | 419 aa | 4.3e−129 |
| barley\|TC126934 Hordeum vulgare | 60% | 191 aa | 4.0e−57 |
| barley\|TC126096 Hordeum vulgare | 62% | 154 aa | 1.8e−53 |
| potato\|TC95923 Solanumtuberosum | 64% | 408 aa | 7.6e−137 |
| sunflower\|BQ978743 Helianthus annuus | 76% | 147 aa | 1.2e−59 |
| sunflower\|BU025789 Helianthus annuus | 53% | 219 aa | 8.2e−55 |
| soybean\|TC189100 Glycine max | 68% | 192 aa | 3.7e−90 |
| soybean\|TC189053 Glycine max | 57% | 285 aa | 2.9e−82 |
| soybean\|TC184911 Glycine max | 77% | 148 aa | 4.3e−60 |
| tomato\|TC129205 L. esculentum | 69% | 193 aa | 1.3e−70 |
| tomato\|AW622540 L. esculentum | 77% | 148 aa | 2.0e−62 |
| tomato\|TC117972 L.esculentum | 65% | 182 aa | 8.5e−61 |
| wheat\|TC154530 Triticum aestivum | 74% | 150 aa | 1.1e−58 |
| wheat\|TC185626 Triticum aestivum | 66% | 141 aa | 1.3e−54 |
| wheat\|TC159965 Triticum aestivum | 59% | 102 aa | 5.4e−39 |
| sorghum\|TC93676 Sorghum bicolor | 60% | 368 aa | 4.3e−114 |
| s_officinarum\|TC11056 Sugarcane | 59% | 203 aa | 7.6e−58 |
| s_officinarum\|CA234860 Sugarcane | 66% | 169 aa | 3.9e−57 |
| s_officinarum\|TC18213 Sugarcane | 62% | 174 aa | 1.4e−56 |
| s_cereale\|BE705622 Rye | 66% | 179 aa | 4.5e−63 |
| pine\|TC38167 Pinus | 59% | 294 aa | 1.2e−86 |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references listed below are incorporated herein by reference to the extent that they supplement, explain, provide a background for, or teach methodology, techniques, and/or compositions employed herein.
U.S. Pat. No. 4,535,060
U.S. Pat. No. 5,302,523
U.S. Pat. No. 5,322,783
U.S. Pat. No. 5,384,253
U.S. Pat. No. 5,464,765
U.S. Pat. No. 5,508,184
U.S. Pat. No. 5,538,877
U.S. Pat. No. 5,538,880
U.S. Pat. No. 5,545,818
U.S. Pat. No. 5,550,318
U.S. Pat. No. 5,563,055
U.S. Pat. No. 5,591,616
U.S. Pat. No. 5,610,042
Abdullah et al., Biotechnology, 4:1087, 1986.
Bailey and Elkan, In: Proceedings of the Second International Conference on Intelligent Systems for Molecular Biology, pp. 28-36, AAAI Press, Menlo Park, Calif., 1994.
Bailey and Gribskov, Bioinformatics, Vol. 14, pp. 48-54, 1998.
Bates, Mol. Biotechnol., 2(2):135-145, 1994.
Battraw and Hall, Theor. App. Genet., 82(2):161-168, 1991.
Bevan et al., Nucleic Acids Research, 11(2):369-385, 1983.
Bhattacharjee et al., J. Plant Bioch. and Biotech. 6, (2):69-73. 1997.
Blancaflor et al., Planta, 217(2):206-17, 2003.
Bouchez et al., EMBO Journal, 8(13):4197-4204, 1989.
Bower et al., Plant Journal, 2:409-416. 1992.
Buckley et al., Eur. J. Pharmacol., 396:141-149, 2000.
Buising and Benbow, Mol Gen Genet, 243(1):71-81. 1994.
Callis et al., Genes Dev., 1: 1183-1200, 1987.
Casa et al., Proc. Natl. Acad. Sci. USA, 90(23):11212-11216, 1993.
Chandler et al., The Plant Cell, 1:1175-1183, 1989.
Chapman et al., Plant Physiol., 120:1157-1164, 1999.
Chapman et al., Plant. Physiol 116: 1163-1168, 1998.
Chapman, Chem. Phys. Lipids, 108:221-230, 2000.
Christou; et al., Proc. Natl. Acad. Sci. USA, 84(12):3962-3966, 1987.
Chu et al., Scientia Sinica, 18:659-668, 1975.
Conkling et al., Plant Physiol., 93:1203-1211, 1990.
DE App. 3642,829
De Block et al., EMBO Journal, 6(9):2513-2518, 1987.
De Block et al., Plant Physiol., 91:694-701, 1989.
De Petrocellis et al., Chemistry and Physics of Lipids 108: 191-209, 2000
Dellaporta et al., In: Chromosome Structure and Function: Impact of New Concepts, 18th Stadler Genetics Symposium, 11:263-282, 1988.
Devane et al., Science 258: 1946-1949, 1992
D'Halluin et al., Plant Cell, 4(12):1495-1505, 1992.
Di Marzo et al., Nature 372: 686-691, 1994
Downward, BMJ, 328(7450):1245-1248, 2004.
EPA 154,204
Ebert et al., 84:5745-5749, Proc. Natl. Acad. Sci. USA, 1987.
Ellis et al., EMBO Journal, 6(11):3203-3208, 1987.
Fire et al., Nature, 391: 806-11, 1998.
Fraley et al., Bio/Technology, 3:629-635, 1985.
Fromm et al., Nature, 319:791-793, 1986.
Gallie et al., The Plant Cell, 1:301-311, 1989.
Gelvin et al., In: Plant Molecular Biology Manual, 1990.
Ghosh-Biswas et al., J. Biotechnol., 32(1):1-10, 1994.
Hagio et al., Plant Cell Rep., 10(5):260-264, 1991.
Hamilton et al., Proc. Natl. Acad. Sci. USA, 93(18):9975-9979, 1996.
Hansen et al., Chem. Phys. Lipids., 108:135-150, 2000.
Haseloff et al., Proc. Natl. Acad. Sci. USA, 94(6):2122-2127, 1997.
He et al., Plant Cell Reports, 14 (2-3):192-196, 1994.
Hensgens et al., Plant Mol. Biol., 22(6):1101-1127, 1993.
Hiei et al., Plant. Mol. Biol., 35(1-2):205-218, 1997.
Hillard et al., J. Neurochem., 64:677-683, 1995.
Hinchee et al., Bio/technol., 6:915-922, 1988.
Hou and Lin, Plant Physiology, 111: 166, 1996.
Hudspeth and Grula, Plant Mol. Biol., 12:579-589, 1989.
Ikuta et al., Bio/technol., 8:241-242, 1990.
Ishidia et al, Nat. Biotechnol., 14(6):745-750, 1996.
Kaeppler et al., Plant Cell Reports 9: 415-418, 1990.
Kaeppler et al., Theor. Appl. Genet., 84(5-6):560-566, 1992.
Katz et al., J. Gen. Microbiol., 129:2703-2714, 1983.
Khanolkar et al., Chemistry and Physics of Lipid 108:37-52, 2000.
Klee et al., Bio-Technology, 3(7):637-642, 1985.
Knittel et al., Plant Cell Reports, 14(2-3):81-86, 1994.
Krogh et al., J. Mol. Biol., 305:567-580, 2001.
Lambert and Di Marzo, Current Med. Chem., 6:663-674, 1999.
Lambert et al., Current Med. Chem., 9:739-755, 2002.
Lawton et al., Plant Mol. Biol. 9:315-324, 1987.
Lazzeri, Methods Mol. Biol., 49:95-106, 1995.
Lee et al., Korean J. Genet., 11(2):65-72, 1989.
Lehner et al., Brief Funct Genomic Proteomic, April;3(1):68-83, 2004.
Lorz et al., Mol Gen Genet, 199:178-182, 1985.
Marcotte et al., Nature, 335:454, 1988.
McCabe, Martinell, Bio-Technology, 11(5):596-598, 1993.
McCormac et al., Euphytica, v. 99 (1) p. 17-25, 1998.
Murakami et al., Mol. Gen. Genet., 205:42-50, 1986.
Murashige and Skoog, Physiol. Plant., 15:473-497, 1962.
Nagatani et al., Biotech. Tech., 11(7):471-473, 1997.
Odell et al., Nature, 313:810-812, 1985.
Ogawa et al., Sci. Rep., 13:42-48, 1973.
Omirulleh et al., Plant Mol. Biol., 21(3):415-428, 1993.
Ow et al., Science, 234:856-859, 1986.
Paria and Dey, Chem. Phys. Lipids, 108:211-220, 2000.
PCT App. WO 94/09699
PCT App. WO 95/06128
PCT App. WO 97/41228
PCT App. WO 97/4103
PCT App. WO 92/17598
Pertwee et al., Eur. J. Pharmacol., 272:73-78, 1995.
Pertwee, Prog. Neurobiol., 63:569-611, 2001.
Potrykus et al., Mol. Gen. Genet., 199:183-188, 1985.
Prasher et al., Biochem. Biophys. Res. Commun., 126(3):1259-1268, 1985.
Reggio P H, Tocris Reviews 10: 1-5, 1999.
Reichel et al., Proc. Natl. Acad. Sci. USA, 93 (12) p. 5888-5893. 1996.
Rhodes et al., Methods Mol. Biol., 55:121-131, 1995.
Ritala et al., Plant Mol. Biol., 24(2):317-325, 1994.
Rogers et al., Methods Enzymol., 153:253-277, 1987.
Sambrook et al., In: Molecular Cloning—A Laboratory Manual (second edition), Cold Spring Harbour Laboratory Press, 1989.
Sarker et al., FEBS Lett., 472:39-44, 2000.
Schmid and Berdyshev, Prostag. Leukotr. Essent. Fatty Acids, 66:363-376, 2002.
Schmid et al., Chem. Phys. Lipids, 121:111-134, 2002.
Schmid et al., Prog. Lipid Res., 29:1-43, 1990.
Sheen et al., Plant Journal, 8(5):777-784, 1995.
Shrestha et al., J. Biol. Chem. 278: 34990-34997, 2003.
Shrestha et al., Plant Physiol., 130:391-401, 2002.
Singsit et al., Transgenic Res., 6(2):169-176, 1997.
Stalker et al., Science, 242:419-422, 1988.
Straus S E, Proc Natl Acad Sci USA 97: 9363-9364, 2000.
Sullivan et al., Mol. Gen. Genet., 215(3):431-440, 1989.
Sutcliffe, Proc. Natl. Acad. Sci. USA, 75:3737-3741, 1978.
Thillet et al., J. Biol. Chem., 263:12500-12508, 1988.
Thomas et al., Plant Sci. 69:189-198, 1990.
Thompson et al., Euphytica, 85(1-3):75-80, 1995.
Thompson et al., The EMBO Journal, 6(9):2519-2523, 1987.
Tian, Sequin, Charest, Plant Cell Rep., 16:267-271, 1997.
Tingay et al., The Plant Journal v. 11 (6) p. 1369-1376. 1997.
Tomes et al., Plant. Mol. Biol. 14(2):261-268, 1990.
Torbet, Rines, Somers, Crop Science, 38(1):226-231, 1998.
Torbet, Rines, Somers, Plant Cell Reports, 14(10):635-640, 1995.
Toriyama et al., Theor Appl. Genet., 73:16, 1986.
Triparthy et al., Plant Physiol 131: 1781-1791, 2003a
Tripathy et al., In Advanced Research on Plant Lipids, 2002: 315-318, N. Murata et al. (eds), 2003b
Tripathy et al., Plant Physiol., 121:1299-1308, 1999.
Tsukada; Kusano; Kitagawa, Plant Cell Physiol., 30(4)599-604, 1989.
Tusnády and Simon, J. Mol. Biol. 283, 489-506, 1998.
Tusnády and Simon, Bioinformatics 17, 849-850, 2001.
Twell et al., Plant Physiol 91:1270-1274, 1989.
Uchimiya et al, Mol. Gen. Genet., 204:204, 1986.
Van der Stelt et al., J. Neurosci, 21:765-8771, 2001.
Van Eck; Blowers; Earle, Plant Cell Reports, 14(5):299-304, 1995.
Vasil et al., Plant Physiol., 91:1575-1579, 1989.
Walker et al., Proc. Natl. Acad. Sci. USA, 84:6624-6628, 1987.
Wang et al., Molecular and Cellular Biology, 12(8):3399-3406, 1992.
Wilson and Nicoll, Science, 296:678-682, 2002.
Yamada et al., Plant Cell Rep., 4:85, 1986.
Yang and Russell, Proc. Natl. Acad. Sci. USA, 87:4144-4148, 1990.
Zheng and Edwards, J. Gen. Virol., 71:1865-1868, 1990.
Zhou et al., Plant Cell Reports, 12(11).612-616, 1993.
Zukowsky et al., Proc. Natl. Acad. Sci. USA, 80:1101-1105, 1983.

What is claimed is:

1. An isolated nucleic acid sequence selected from the group consisting of:
(a) a nucleic acid sequence encoding the polypeptide of SEQ ID NO:2 or SEQ ID NO:4;
(b) a nucleic acid sequence comprising the sequence of SEQ ID NO:1 or SEQ ID NO:3;
(c) a nucleic acid sequence hybridizing to SEQ ID NO 1 or SEQ ID NO:3 under conditions of 5×SSC, 50% formamide and 42° C.; and
(d) a nucleic acid sequence encoding a polypeptide having at least 90% sequence identity to the polypeptide of SEQ ID NO:2 or SEQ ID NO:4.

2. An isolated nucleic acid sequence encoding a plant N-acylethanolamine (NAE) binding protein, wherein the nucleic acid is operably linked to a heterologous promoter, wherein the isolated nucleic acid sequence encodes the polypeptide encoded by a nucleic acid sequence selected from the group consisting of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 and SEQ ID NO:29.

3. The isolated nucleic acid sequence of claim 2, further defined as comprising a nucleic acid sequence selected from the group consisting of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28 and SEQ ID NO:29.

4. A recombinant vector comprising the isolated nucleic acid sequence of claim 1 or an antisense or RNAi oligonucleotide thereof.

5. The recombinant vector of claim 4, further comprising at least one additional sequence chosen from the group consisting of: a regulatory sequence, a selectable marker, a leader sequence and a terminator.

6. The recombinant vector of claim 5, wherein the additional sequence is a heterologous sequence.

7. The recombinant vector of claim 4, wherein the promoter is a developmentally-regulated, organelle-specific, inducible, tissue-specific, constitutive, cell-specific, seed specific, or germination-specific promoter.

8. The recombinant vector of claim 4, defined as an isolated expression cassette.

9. An isolated polypeptide comprising the amino acid sequence of SEQ ID NO:2, or a fragment thereof having NAE binding protein activity.

10. A transgenic plant transformed with a selected DNA comprising the nucleic acid sequence of claim 1.

11. The transgenic plant of claim 10, further defined as an R₀ transgenic plant.

12. The transgenic plant of claim 10, further defined as a progeny plant of any generation of an R₀ transgenic plant, wherein said transgenic plant has inherited said selected DNA from said R₀ transgenic plant.

13. A seed of the transgenic plant of claim 10, wherein said seed comprises said selected DNA.

14. A host cell transformed with a selected DNA comprising the nucleic acid sequence of claim 1.

15. The host cell of claim 14, wherein said host cell expresses a protein encoded by said selected DNA.

16. The host cell of claim 14, wherein the cell has inherited said selected DNA from a progenitor of the cell.

17. The host cell of claim 14, wherein the cell has been transformed with said selected DNA.

18. The host cell of claim 14, wherein said host cell is a plant cell.

19. A method of altering the perception of N-acylethanolamine (NAE) in a plant comprising introducing into the plant the isolated nucleic acid sequence of claim 1, wherein the isolated nucleic acid sequence is expressed in the plant.

20. The method of claim 19, wherein the isolated nucleic acid sequence is in sense orientation.

21. The method of claim 19, wherein the isolated nucleic acid sequence is in antisense orientation.

22. The method of claim 19, wherein the isolated nucleic acid is in sense and antisense orientation.

23. The method of claim 19, wherein N-acylethanolamine perception is increased in the plant relative to a plant of the same genotype lacking the isolated nucleic acid.

24. The method of claim 19, wherein N-acylethanolamine perception is decreased in the plant relative to a plant of the same genotype lacking the isolated nucleic acid.

25. The method of claim 19, wherein the growth of the plant is increased or decreased as a result of the expression of the isolated nucleic acid sequence.

26. The method of claim 19, wherein the isolated nucleic acid is up-regulated and the stress tolerance of the plant is increased as a result of the expression of the isolated nucleic acid sequence.

27. The method of claim 19, wherein introducing the isolated nucleic acid comprises plant breeding.

28. The method of claim 19, wherein introducing the isolated nucleic acid comprises genetic transformation.

29. The method of claim 19, wherein the pathogen perception of the plant is increased as a result of the expression of the isolated nucleic acid.

30. A method of altering the perception of N-acylethanolamine (NAE) in a plant comprising introducing into the plant the isolated nucleic acid sequence of claim 2.

31. A method of producing food for human or animal consumption comprising:
(a) obtaining the plant of claim 10;
(b) growing said plant under plant growth conditions to produce plant tissue from the plant; and
(c) preparing food for human or animal consumption from said plant tissue.

32. The method of claim 31, wherein preparing food comprises harvesting said plant tissue.

33. The method of claim 32, wherein said food is starch, protein, meal, flour or grain.
